(19) United States
(12) Patent Application Publication
(10) Pub. No.: US 2026/0158178 A1
(43) Pub. Date: Jun. 11, 2026

(54) 19F MAGNETIC RESONANCE IMAGING AGENTS AND METHODS OF USING THEREOF

(71) Applicant: Kuva Labs Inc., Houston, TX (US)

(72) Inventors: Andrew Hopkins, Houston, TX (US); Thomas Hopkins, Houston, TX (US)

(21) Appl. No.: 19/405,896

(22) Filed: Dec. 2, 2025

Related U.S. Application Data
(60) Provisional application No. 63/729,054, filed on Dec. 6, 2024.

(51) Int. Cl. A61K 51/06; C08G 65/34

(52) U.S. Cl. CPC A61K 51/06; C08G 65/34; A61K 2121/00; A61K 2123/00

(57) ABSTRACT

Pharmaceutical compositions containing ¹⁹F-containing polymers and methods of using thereof for imaging applications are described herein. Systems and kits containing such pharmaceutical compositions are also described. For instance, the ¹⁹F-containing polymers can be used to image a target tissue and may be used analyze the size, shape, position, and/or the composition of the target tissue and/or to treat a disease or condition.

### PRIORITY APPLICATION

This application claims the benefit of U.S. Provisional Patent Application No. 63/729,054, filed Dec. 6, 2024, the disclosure of which is incorporated by reference herein in its entirety.

### FIELD OF THE INVENTION

The disclosed invention is generally in the field of ¹⁹F-containing polymers and methods of making and using thereof, such as for agents for magnetic resonance imaging.

### BACKGROUND OF THE INVENTION

¹⁹F nuclear magnetic resonance (NMR) agents are specialized compounds used in NMR and magnetic resonance imaging (MRI) to provide highly sensitive, fluorine-specific contrast. Fluorine-19 (¹⁹F) is suited for NMR studies due to its high gyromagnetic ratio, 100% natural abundance, and sensitivity comparable to hydrogen (¹H). Unlike ¹H NMR, ¹⁹F NMR agents can produce background-free signal, since fluorine is rarely present in biological tissues.

Despite their potential, ¹⁹F NMR agents face several challenges, particularly concerning their poor water solubility. This limitation affects their bioavailability, delivery efficiency, and compatibility with biological systems. For instance, known ¹⁹F NMR agents, especially those derived from perfluorocarbons or other heavily fluorinated compounds, are hydrophobic and exhibit low solubility in aqueous environments. Poor solubility limits their dispersibility in blood or other physiological fluids, reducing their effectiveness in vivo and making them challenging to administer intravenously. Such insoluble agents can, for instance, have uneven biodistribution due to their poor water solubility, reducing the reliability and resolution of ¹⁹F NMR/MRI imaging. Delivery of such insoluble ¹⁹F NMR agents to specific tissues or cells is also difficult.

Thus, there remains a need for ¹⁹F imaging agents which addresses and overcomes the issues of known ¹⁹F imaging agents, such as improving the solubility of ¹⁹F imaging agents, and/or improving the reliability and/or resolution of ¹⁹F NMR/MRI imaging in tissue of a subject.

Accordingly, it is an object of the invention to provide improved ¹⁹F imaging agents.

It is a further object of the invention to provide methods of making such improved ¹⁹F imaging agents.

It is yet a further object of the invention to provide methods of using such improved ¹⁹F imaging agents for magnetic resonance imaging applications.

### BRIEF SUMMARY OF THE INVENTION

Described herein are ¹⁹F-containing polymers and methods of making and using thereof. The ¹⁹F-containing polymers exhibit improved solubility properties and can provide improved ¹⁹F NMR/MRI imaging.

In one non-limiting instance, a ¹⁹F-containing polymer includes:
a polymer backbone formed of a plurality of polymer repeat units;
wherein at least about 1% and up to 99% of the polymer repeat units of the plurality include one or more ¹⁹F-atoms or ¹⁹F-containing groups covalently associated thereto; and
wherein the ¹⁹F-containing polymer is hydrophilic and is soluble or substantially soluble in one or more pharmaceutically acceptable carriers. Such ¹⁹F-containing polymers can have various chemical structures as shown in the various formulas described below.

Various forms of ¹⁹F-containing polymers are possible, such as where the polymers can have both targeting and active agent(s) covalently bonded thereto, only targeting agent(s) covalently bonded thereto, or only active agent(s) covalently bonded thereto. The targeting and active agents can be those described above. In some instances, one or both of targeting and active agent(s) are not covalently attached to the ¹⁹F-containing polymers and the one or both of targeting and active agent(s) are co-administered with the ¹⁹F-containing polymers, as described in the systems below. In still other instances, one or both of targeting and active agent(s) are non-covalently associated to the ¹⁹F-containing polymers and the one or both of targeting and active agent(s) are co-administered with the ¹⁹F-containing polymers, as described in the systems below.

In addition to linear polymers, a ¹⁹F-containing polymer may also be branched ¹⁹F-containing polymers, such as multi-armed ¹⁹F-containing polymers. Thus, in some non-limiting instances, a ¹⁹F-containing polymer includes:
a polymer core including a plurality of arms, wherein each arm includes a plurality of polymer repeat units;
wherein at least about 1% and up to about 99% of the polymer repeat units of the plurality include one or more ¹⁹F-atoms or ¹⁹F-containing groups covalently associated thereto; and
wherein the ¹⁹F-containing polymer is hydrophilic and is soluble or substantially soluble in one or more pharmaceutically acceptable carriers.

For the various ¹⁹F-containing polymers described above, whether linear or branched, these polymers can have any suitable molecular weight. In some instances, the ¹⁹F-containing polymer has a weight-average molecular weight in a range from about 500 to 200,000 Daltons, 500 to 175,000 Daltons, 500 to 150,000 Daltons, 500 to 125,000 Daltons, 500 to 100,000 Daltons, 500 to 75,000 Daltons, 500 to 50,000 Daltons, 500 to 25,000 Daltons, 500 to 10,000 Daltons, 500 to 5,000 Daltons, 1,000 to 75,000 Daltons, 1,000 to 50,000 Daltons, 1,000 to 25,000 Daltons, 1,000 to 10,000 Daltons, or 1,000 to 5,000 Daltons, as well as individual values or sub-ranges contained within the aforementioned ranges. In some instances, the ¹⁹F-containing polymer has a hydrodynamic diameter of less than about 50 nm, 40 nm, 30 nm, 20 nm, 10 nm, or 5 nm. In some instances, the ¹⁹F-containing polymer has a hydrodynamic diameter in a range from about 5 nm to 50 nm, as well as individual values or sub-ranges contained within the aforementioned range.

Pharmaceutical compositions are also disclosed herein which include the ¹⁹F-containing polymers described herein. The pharmaceutical composition includes an effective amount of the ¹⁹F-containing polymers for purposes of ¹⁹F magnetic resonance imaging.

In one non-limiting instance, a pharmaceutical composition includes:
a plurality of the ¹⁹F-containing polymers, as described herein; and
one or more pharmaceutically acceptable carriers;
wherein the plurality of the ¹⁹F-containing polymers are solubilized or substantially solubilized in the one or more pharmaceutically acceptable carriers.

In some instances, such a pharmaceutical composition can be combined with one or more additional compositions to form a system. For example, in one non-limiting instance, such a system can include:
a pharmaceutical composition including:
   a plurality of the ¹⁹F-containing polymers as described herein; and
   one or more pharmaceutically acceptable carriers;
   wherein the plurality of the ¹⁹F-containing polymers are solubilized or substantially solubilized in the one or more pharmaceutically acceptable carriers;
and
at least one targeting composition including:
a plurality of agents for targeted delivery of the plurality of the ¹⁹F-containing polymers of the pharmaceutical composition to a target tissue in a subject.

The ¹⁹F-containing polymers and compositions and systems containing thereof can be used as a diagnostic and imaging tool for various biological applications. In one non-limiting instance, a method of imaging a target tissue in a subject includes the steps of:
(i) administering a pharmaceutical composition described herein to a subject;
(ii) allowing an effective period of time for the plurality of the ¹⁹F-containing polymers of the pharmaceutical composition to accumulate in the target tissue of the subject; and
(iii) imaging by ¹⁹F magnetic resonance imaging the plurality of the ¹⁹F-containing polymers following step (ii);
   wherein the imaging permits visualizing the target tissue and optionally may be used to analyze size, shape, position, and composition of the target tissue.

In another non-limiting instance, a method of imaging a target tissue in a subject includes the steps of:
(i′) administering a system described herein (i.e., including a pharmaceutical compositions and a targeting composition) to a subject;
(ii′) allowing an effective period of time for the plurality of the ¹⁹F-containing polymers of the pharmaceutical composition to accumulate in a target tissue of the subject; and
(iii′) imaging by ¹⁹F magnetic resonance imaging the plurality of the ¹⁹F-containing polymers following step (ii′);
   wherein the imaging permits visualizing the target tissue and optionally may be used to analyze size, shape, position, and composition of the target tissue.

In still another non-limiting instance, a method of treating or ameliorating a disease or condition in a subject in need thereof includes the steps of:
(a) administering a pharmaceutical composition or a system, such as those described herein, to a subject;
(b) allowing an effective period of time for the plurality of the ¹⁹F-containing polymers of the pharmaceutical composition to accumulate in a target tissue of the subject; and
(c) imaging by ¹⁹F magnetic resonance imaging the plurality of the ¹⁹F-containing polymers following step (b);
   wherein the imaging permits visualizing the target tissue and optionally may be used to analyze size, shape, position, and composition of the target tissue;
wherein the ¹⁹F-containing polymers include at least one active agent associated thereto selected from the group consisting of a therapeutic agent, a chemotherapeutic agent, a photosensitizer (such as IR700 or a phthalocyanine dye), and combinations thereof.

Kits useful for practicing the methods described above, and the various pharmaceutical and other compositions used therein, are also disclosed herein. For example, kits for ¹⁹F-based imaging applications can include at least one or more of the pharmaceutical compositions or systems including the ¹⁹F-containing polymer(s) and optionally a targeting composition.

### BRIEF DESCRIPTION OF THE DRAWINGS

The accompanying drawings illustrate several embodiments of the disclosed compositions and methods thereof which together with the description serve to explain the principles of the disclosed methods and compositions.

FIG. 1 shows an exemplary non-limiting polymer synthesis scheme for forming a ¹⁹F-substituted polyethylene glycol (PEG) polymer, where the polymer may be prepared via an anionic polymerization using nitrogen purged dimethylsulfoxide (DMSO) and a base (or water) as an initiator. The reaction may be terminated by addition of an acid—the reaction time, monomer feed concentration, temperature, and base concentration may be used to control the molecular weight of the final polymer product. Variables R₁, R₂, R₃, R₄, and n are as defined below for Formula I.

FIG. 2 shows an exemplary non-limiting polymer synthesis scheme for forming a ¹⁹F-substituted polyethylene imine (PEI) polymer, where the polymer may be prepared via an anionic polymerization using nitrogen purged dimethylsulfoxide (DMSO) and a base as an initiator. The reaction may be terminated by addition of an acid—the reaction time, monomer feed concentration, temperature, and base concentration may be used to control the molecular weight of the final polymer product. Variables R₁, R₂, R₃, R₄, and n are as defined below for Formula II.

FIG. 3 shows an exemplary non-limiting polymer synthesis scheme for forming a ¹⁹F-substituted poly(meth)acrylate/acid, where the polymer may be prepared via a radical polymerization using nitrogen purged water and ammonium persulfate (APS) as an initiator. The reaction may be terminated by allowing oxygen into the reaction environment—the reaction time, monomer feed concentration, temperature, and APS concentration may be used to control the molecular of the final polymer product. Variables R₁, R₂, R₃, R₄, and n are as defined below for Formula III.

FIG. 4 shows an exemplary non-limiting polymer synthesis scheme for forming a ¹⁹F-substituted poly(meth)acrylamide, where the polymer may be prepared via a radical polymerization using nitrogen purged water and ammonium persulfate (APS) as an initiator. The reaction may be terminated by allowing oxygen into the reaction environment—the reaction time, monomer feed concentration, temperature, and APS concentration may be used to control the molecular of the final polymer product. Variables R₁, R₂, R₃, R₄, and n are as defined below for Formula IV.

FIG. 5 shows the ¹⁹F NMR spectrum of monomer 3,3,3-trifluoropropane-1,2-diol of Example 1.

FIG. 6 shows a graph of the T₂ of 3,3,3-trifluoropropane-1,2-diol based on ¹⁹F NMR data obtained in Example 1.

### DETAILED DESCRIPTION OF THE INVENTION

¹⁹F-containing polymers and methods of making and using thereof are described herein. Such ¹⁹F-containing polymers exhibit improved solubility properties and thereby provide improved ¹⁹F NMR/MRI imaging.

### I. Definitions

“Pharmaceutically acceptable carrier” refers a substance or combination of substances used in compositions to deliver one or more ingredients, such as tracers, safely and effectively to a subject without causing adverse reactions or diminishing the efficacy or stability of the ingredients. Pharmaceutically acceptable carriers meet standards set by regulatory bodies, such as the U.S. Food and Drug Administration (FDA), to ensure they are safe for human use. These standards often include being non-toxic, non-reactive, and compatible with the dosage form and route of administration.

“Alcohol” refers to the group —R—OH, where R is absent or R is an alkyl group. It is understood that the —OH group may be on any position of the alkyl group R and that more than one-OH group may be present. Unless specified otherwise the term alcohol embraces salt forms of the alcohol, such as —R—O⁻M⁺, where M⁺ is a counter cation, such as Na⁺ or K⁺. In some instances, the alcohol is a C₁-C₆ alcohol.

“Amine group” refers to —NH₂ or primary, secondary, or tertiary amines by replacing the hydrogen atoms thereon, for example, organic substituents. A primary amine contains one organic substituent and two hydrogen atoms attached to the nitrogen atom (e.g., R—NH₂, where R is an organic group like alkyl group). A secondary amine contains two organic substituents and one hydrogen atom attached to the nitrogen (e.g., —NH—R, where R can be the same or different organic groups, such as alkyl groups). A tertiary amine has three organic substituents and no hydrogen atoms attached to the nitrogen (e.g., —N(R)₂, where R can be the same or different organic groups, such as alkyl groups). Quaternary ammonium ions of the amines described are also encompassed by the term creating a positively charged species, which may exist as a salt forms having an X counter anion, such as Cl⁻, associated thereto. The term “amine group” also encompasses related derivatives, such as salts (e.g., amine hydrochloride), protected amines (e.g., carbamate-protected), or functional equivalents (e.g., amides).

“Carboxylic acid” refers to the group-COOH. Unless specified otherwise the term carboxylic acid embraces both the free acid and carboxylate salt.

“Alkyl” (—R) refers to saturated or unsaturated aliphatic groups, including straight-chain alkyl, alkenyl, or alkynyl groups, branched-chain alkyl, alkenyl, or alkynyl groups, cycloalkyl, cycloalkenyl, or cycloalkynyl (alicyclic) groups, alkyl substituted cycloalkyl, cycloalkenyl, or cycloalkynyl groups, and cycloalkyl substituted alkyl, alkenyl, or alkynyl groups. Unless otherwise indicated, a straight chain or branched chain alkyl has 30 or fewer carbon atoms in its backbone (e.g., C₁-C₃₀ for straight chain, C₃-C₃₀ for branched chain), more preferably 20 or fewer carbon atoms, more preferably 12 or fewer carbon atoms, and most preferably 8 or fewer carbon atoms. In some instances, the chain has 1-6 carbons. Likewise, cycloalkyls can have from 3-10 carbon atoms in their ring structure, and more preferably have 5, 6 or 7 carbons in the ring structure. The ranges provided above are inclusive of all values between the minimum value and the maximum value. The term “alkyl” includes both “unsubstituted alkyls” and “substituted alkyls”, the latter of which refers to alkyl moieties having one or more substituents replacing a hydrogen on one or more carbons of the hydrocarbon backbone. Such substituents include, but are not limited to, halogen, hydroxyl, ether, ester, carbonyl (such as a carboxyl, alkoxycarbonyl, formyl, or an acyl), thiocarbonyl (such as a thioester, thionoester, a thioacetate, or a thioformate), alkoxyl, phosphoryl, phosphate, phosphonate, a phosphinate, amino, amido, amidine, imine, cyano, isocyano, nitro, azido, sulfhydryl, alkylthio, sulfate, sulfonate, sulfamoyl, sulfonamido, sulfonyl, heterocyclyl, or an aromatic or heteroaromatic moiety. Exemplary alkyl radicals include, without limitation, methyl, ethyl, i-propyl, n-propyl, i-butyl, n-butyl, t-butyl, sec-butyl, i-pentyl, n-pentyl, sec-pentyl, neopentyl, n-hexyl, i-hexyl, and sec-hexyl.

Unless the number of carbons is otherwise specified, “lower alkyl” as used herein means an alkyl group, as defined above, but having from one to ten carbons, more preferably from one to six carbon atoms, in its backbone structure. Likewise, “lower alkenyl” and “lower alkynyl” have similar chain lengths. Preferred alkyl groups are lower alkyls. The alkyl groups may also contain one or more heteroatoms within the carbon backbone. Examples include oxygen, nitrogen, sulfur, and combinations thereof. In certain embodiments, the alkyl group contains between one and four heteroatoms.

“Carbonyl group” refers to moieties that can be represented by the general formula:

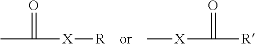

wherein X is a bond, or represents an oxygen or a sulfur, and R represents a hydrogen, a substituted or unsubstituted alkyl, substituted or unsubstituted alkenyl, substituted or unsubstituted alkynyl, substituted or unsubstituted cycloalkyl, substituted or unsubstituted aryl, or substituted or unsubstituted heteroaryl, —(CH₂)ₘ—R″; wherein R′ represents a hydrogen, substituted or unsubstituted alkyl, substituted or unsubstituted alkenyl, substituted or unsubstituted alkynyl, substituted or unsubstituted cycloalkyl, substituted or unsubstituted aryl, or substituted or unsubstituted heteroaryl or —(CH₂)ₘ—R″; wherein R″ represents a hydroxy group, substituted or unsubstituted carbonyl group, an aryl, a cycloalkyl, a heterocycle, or a polycycle; and m is zero or an integer ranging from 1 to 8. Where X is oxygen and R is defined as above, the moiety can be referred to as a “carboxyl group.” When X is oxygen and R is hydrogen, the formula represents a “carboxylic acid group.” Where X is oxygen and R′ is hydrogen, the formula represents a “formate group.” Where X is oxygen and R or R′ is not hydrogen, the formula represents an “ester group.” In general, where the oxygen atom of the above formula is replaced by a sulfur atom, the formula represents a “thiocarbonyl group.” Where X is sulfur and R or R′ is not hydrogen, the formula represents a “thioester group.” Where X is sulfur and R is hydrogen, the formula represents a “thiocarboxylic acid group.” Where X is sulfur and R′ is hydrogen, the formula represents a “thioformate group.” Where X is a bond and R is not hydrogen, the above formula represents a “ketone group.” Where X is a bond and R is hydrogen, the above formula represents an “aldehyde group.” The term “substituted carbonyl” refers to a carbonyl, as defined above, wherein one or more hydrogen atoms in R, R′ or a group to which the moiety is attached, are independently substituted with suitable substituents, as defined below.

An “aryl group” (—Ar) refers to a structure made up of 6 to 30 carbon atoms, 6 to 18 carbon atoms, which is formed from one aromatic ring or a plurality of fused aromatic rings. Exemplary aryl groups are, without limitation, phenyl, naphthyl, anthracenyl, or phenanthrenyl. Aryl groups may be unsubstituted, where all carbon atoms which are substitutable bear hydrogen atoms. Alternatively, they may be substituted at one, greater than one, or at all substitutable positions therein. Suitable exemplary substituents include, without limitation, alkyl groups, such as alkyl groups having 1 to 8 carbon atoms, which may be selected from methyl, ethyl, i-propyl or t-butyl, aryl groups (such as C₆-aryl groups, which may be substituted or unsubstituted), heteroaryl groups (which may include at least one nitrogen atom, such as pyridyl groups), alkenyl groups (which may include one double bond and 1 to 8 carbon atoms), or groups with electron donating or electron accepting ability. Groups with electron donating ability are understood to mean groups which have a positive inductive (+I) and/or positive mesomeric (+M) effect, and groups with electron accepting ability are understood to mean groups which have a negative inductive (—I) and/or negative mesomeric (-M) effect. Suitable groups with donor or acceptor action are halogens (such as F, Cl, Br), alkoxy groups, aryloxy groups, carbonyl groups, ester groups, amine groups, amide groups, CH₂F groups, CHF₂ groups, CF₃ groups, CN groups, thio groups, or SCN groups.

“Halogen” refers to fluorine, chlorine, bromine, or iodine.

The term “substituted” refers to all permissible substituents of the compounds described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, preferably 1-14 carbon atoms, and optionally include one or more heteroatoms such as oxygen, sulfur, or nitrogen grouping in linear, branched, or cyclic structural formats. Representative substituents include alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aryloxy, substituted aryloxy, alkylthio, substituted alkylthio, phenylthio, substituted phenylthio, arylthio, substituted arylthio, cyano, isocyano, isocyanato, substituted isocyanato, isothiocyanato, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, amino acid, peptide, and polypeptide groups.

Heteroatoms, such as nitrogen, may have hydrogen substituents and/or any permissible substituents of organic compounds described herein that satisfy the valences of the heteroatoms. It is understood that “substitution” or “substituted” includes the implicit proviso that such substitution is in accordance with permitted valence of the substituted atom and the substituent, and that the substitution results in a stable compound, i.e., a compound that does not spontaneously undergo transformation such as by rearrangement, cyclization, elimination, etc.

“Polymer” refers to a molecule containing at least 5 or more monomer repeat units.

The term “effective period of time” or “effective amount of time” refers to the amount of time which is sufficient to cause a desired condition, effect, or outcome to occur. It is typically the minimum span of time required to effectively produce the intended result.

“Contrast” refers to the relative difference of signal intensities in two adjacent regions (i.e., pixels or voxels) of an image. Due to the T₁ and T₂ relaxation properties in magnetic (MRI) resonance imaging, differentiation between various tissues is possible. Contrast is affected by not only the T₁ and T₂ values of specific tissues and contrast agents, but also the differences in the magnetic field strength, temperature changes, and various other factors. In some instances, contrast can be controlled based on the selection of appropriate pulse sequences (spin echo, inversion recovery, gradient echo, turbo sequences and slice profile).

The terms “treating” or “ameliorating” in the context of a disease or disorder means to ameliorate, reduce or otherwise stop a disease, disorder, or condition from occurring or progressing in an animal which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected, such as treating the pain of a subject by administration of an analgesic agent even though such agent does not treat the cause of the pain. Desirable effects of treatment include decreasing the rate of disease progression, ameliorating, or palliating the disease state, and remission or improved prognosis. For example, a subject is “treated” if one or more symptoms associated with a cancer is mitigated or eliminated, including, but not limited to, increasing the quality of life of those suffering from the cancer, decreasing the dose of other medications required to treat the cancer, delaying the progression of the cancer, and/or prolonging survival of subject(s).

The term “hydrophilic” refers to the property of having an affinity for water. For example, hydrophilic polymers (or hydrophilic polymer segments) are polymers (or polymer segments) which are primarily soluble in water and aqueous solutions and/or have a tendency to absorb water. In general, the more hydrophilic a polymer is, the more that polymer tends to dissolve in, mix with, or be wetted by water. A ¹⁹F-containing polymer is considered hydrophilic when at least about 1 g of the polymer is fully solvated/dissolved in 1 liter of water or an aqueous solution to provide a 0.1 wt %. solution at standard temperature and pressure.

Numerical ranges disclosed herein disclose individually each possible number in such range, as well as any sub-ranges and combinations of sub-ranges encompassed therein. For example, a carbon range (e.g., C₁-C₁₀) is intended to disclose individually every possible carbon value and/or sub-range encompassed within. For example, a carbon length range of C₁-C₁₀ discloses C₁, C₂, C₃, C₄, C₅, C₆, C₇, C₈, C₉, and C₁₀, as well as discloses sub-ranges encompassed within, such as C₂-C₉, C₃-C₈, C₁-C₅, etc. Similarly, an integer value range of 1-10 discloses the individual values of 1, 2, 3, 4, 5, 6, 7, 8, 9, and 10, as well as sub-ranges encompassed within. Further, a concentration range or weight percent range, such as from 1% to 2% by weight of the formulation discloses, the individual values and fractions thereof, such as 1%, 1.1%, 1.2%, 1.32%, 1.48% etc., as well as sub-ranges encompassed within.

Use of the term “about” is intended to describe values either above or below the stated value in a range of approx. +/−10%; in other instances the values may range in value either above or below the stated value in a range of approx. +/−5%; in other instances the values may range in value either above or below the stated value in a range of approx. +/−2%; in other instances the values may range in value either above or below the stated value in a range of approx. +/−1%. The preceding ranges are intended to be made clear by context, and no further limitation is implied.

All methods described herein can be performed in any suitable order unless otherwise indicated herein or otherwise clearly contradicted by context.

### II. ¹⁹F-Containing Polymers and Pharmaceutical Compositions Thereof

There is a need for water soluble polymers having fluorinated groups which are compatible with biological systems and can serve as a class of directly magnetic resonance (MR) imageable agents with essentially zero background interferences, due to the absence of endogenous ¹⁹F in biological systems. Combined with suitable molecular weights, MR imaging sequencing, and targeting, such ¹⁹F-based polymeric agents can be used to provide images with enhanced contrast and/or clarity and high specificity for visualizing tissues. These agents can be modified (such as conjugated) with active agents to target and treat tissues, such as tumors. In some other instances, these agents can be co-administered with active agents to target and/or treat a disease.

Various ¹⁹F-containing polymers that can function as ¹⁹F imaging agents are described below.

In some non-limiting instances, a ¹⁹F-containing polymer includes:
a polymer backbone formed of a plurality of polymer repeat units;
wherein at least about 1% and up to 99% of the polymer repeat units of the plurality include one or more ¹⁹F-atoms or ¹⁹F-containing groups covalently associated thereto; and
wherein the ¹⁹F-containing polymer is hydrophilic and is soluble or substantially soluble in one or more pharmaceutically acceptable carriers. “Substantially soluble,” refers to the ¹⁹F-containing polymer being fully dissolved in one or more pharmaceutically acceptable carriers at standard temperature and pressure and at weight percentages in ranges of about 0.1 wt. % to 20 wt. %, 0.1 wt. % to 15 wt. %, 0.1 wt. % to 10 wt. %, or 0.1 wt. % to 5 wt. %, as well as individual values or sub-ranges contained within the aforementioned ranges. The one or more pharmaceutically acceptable carriers are typically aqueous carriers. In some instances, the ¹⁹F-containing polymer is more soluble in a polar solvent, such as an alcohol, but still demonstrates solubility in water at the aforementioned weight percentages.

In some instances, in addition to or in place of at least about 1% and up to 99% of the polymer repeat units of the plurality including one or more ¹⁹F-atoms or ¹⁹F-containing groups covalently associated thereto, the polymer repeat units of the plurality include the one or more ¹⁹F-atoms or ¹⁹F-containing groups covalently associated thereto and the molar percentage of fluorine per repeat unit is less than about 75%, 70%, 60%, 50%, 40%, 30%, 20%, 10%, 5%, or 1%; or the molar percentage of fluorine per repeat unit is in a range from about 1% to 75%, as well as individual values or sub-ranges contained within the aforementioned range. The percentage of fluorine per repeat unit is calculated by the total molar mass of fluorine atom(s) present on each repeat unit divided by the total molar mass of all of the atoms of the repeat unit times 100.

Such ¹⁹F-containing polymers can have various chemical structures. In one instance, the polymer backbone of a ¹⁹F-containing polymer has a structure according to the following formula:

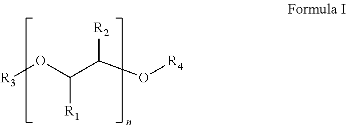

wherein n is an integer value in a range from 5 to 2,000;
wherein at least some, but not all, of the R₁ and R₂ groups are the one or more ¹⁹F-atoms or ¹⁹F-containing groups and the remaining R₁ and R₂ groups are each independently selected from the group consisting of hydrogen, a halogen, an alkyl group, an aryl group, an alcohol, an amine, an acid (such as a carboxylic acid), a carbonyl group, an ester, and a ketone; and
wherein R₃ and R₄ are terminal end groups or structures and wherein R₃ and R₄ do not include ¹⁹F-atoms or ¹⁹F-containing groups.

In some instances, the ¹⁹F-containing polymer of Formula I has an extended structure as follows:

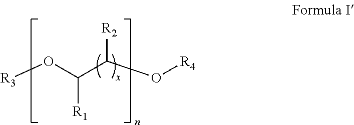

wherein n is an integer value in a range from 5 to 2,000;
wherein x is an integer value in a range from 2 to 15;
wherein at least some, but not all, of the R₁ and R₂ groups are the one or more ¹⁹F-atoms or ¹⁹F-containing groups and the remaining R₁ and R₂ groups are each independently selected from the group consisting of hydrogen, a halogen, an alkyl group, an aryl group, an alcohol, an amine, an acid (such as a carboxylic acid), a carbonyl group, an ester, and a ketone; and
wherein R₃ and R₄ are terminal end groups or structures and wherein R₃ and R₄ do not include ¹⁹F-atoms or ¹⁹F-containing groups.

In a second instance, the polymer backbone of a ¹⁹F-containing polymer has a structure according to the following formula:

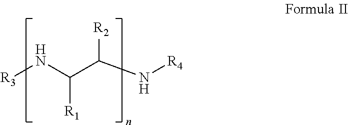

wherein n is an integer value in a range from 5 to 2,000;
wherein at least some, but not all, of the R₁ and R₂ groups are the one or more ¹⁹F-atoms or ¹⁹F-containing groups and the remaining R₁ and R₂ groups are each independently selected from the group consisting of hydrogen, a halogen, an alkyl group, an aryl group, an alcohol, an amine, an acid (such as a carboxylic acid), a carbonyl group, an ester, and a ketone; and
wherein R₃ and R₄ are terminal end groups or structures and wherein the R₃ and R₄ do not include ¹⁹F-atoms or ¹⁹F-containing groups.

In some instances, the ¹⁹F-containing polymer of Formula II has an extended structure as follows:

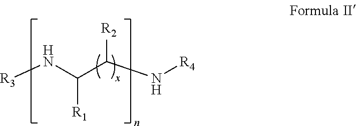

wherein n is an integer value in a range from 5 to 2,000;
wherein x is an integer value in a range from 2 to 15;
wherein at least some, but not all, of the R₁ and R₂ groups are the one or more ¹⁹F-atoms or ¹⁹F-containing groups and the remaining R₁ and R₂ groups are each independently selected from the group consisting of hydrogen, a halogen, an alkyl group, an aryl group, an alcohol, an amine, an acid (such as a carboxylic acid), a carbonyl group, an ester, and a ketone; and
wherein R₃ and R₄ are terminal end groups or structures and wherein R₃ and R₄ do not include ¹⁹F-atoms or ¹⁹F-containing groups.

In a third instance, the polymer backbone of a ¹⁹F-containing polymer has a structure according to the following formula:

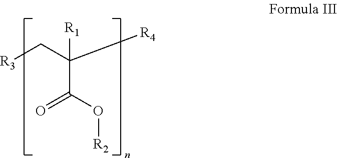

wherein n is an integer value in a range from 5 to 2,000;
wherein at least some, but not all, of the R₁ and R₂ groups are the one or more ¹⁹F-atoms or ¹⁹F-containing groups and the remaining R₁ and R₂ groups are each independently selected from the group consisting of hydrogen, a halogen, an alkyl group, an aryl group, an alcohol, an amine, an acid (such as a carboxylic acid), a carbonyl group, an ester, and a ketone; and
wherein R₃ and R₄ are terminal groups or structures and wherein the R₃ and R₄ do not include ¹⁹F-atoms or ¹⁹F-containing groups.

In a fourth instance, the polymer backbone of a ¹⁹F-containing polymer has a structure according to the following formula:

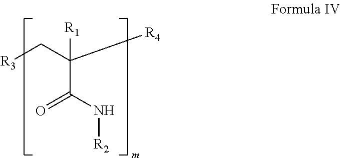

wherein n is an integer value in a range from 5 to 2,000;
wherein at least some, but not all, of the R₁ and R₂ groups are the one or more ¹⁹F-atoms or ¹⁹F-containing groups and the remaining R₁ and R₂ groups are each independently selected from the group consisting of hydrogen, a halogen, an alkyl group, an aryl group, an alcohol, an amine, an acid (such as a carboxylic acid), a carbonyl group, an ester, and a ketone; and
wherein R₃ and R₄ are terminal groups or structures and wherein the R₃ and R₄ do not include ¹⁹F-atoms or ¹⁹F-containing groups.

In some instances, the ¹⁹F-containing polymer of Formulas III and IV have extended structures as follows:

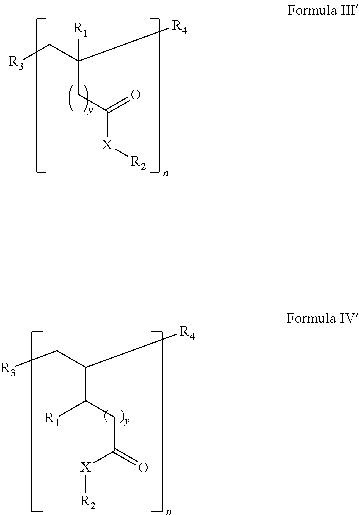

wherein X is —O— or —N(H)—
wherein n is an integer value in a range from 5 to 2,000;
wherein y is an integer value in a range from 2 to 15;
wherein at least some, but not all, of the R₁ and R₂ groups are the one or more ¹⁹F-atoms or ¹⁹F-containing groups and the remaining R₁ and R₂ groups are each independently selected from the group consisting of hydrogen, a halogen, an alkyl group, an aryl group, an alcohol, an amine, an acid (such as a carboxylic acid), a carbonyl group, an ester, and a ketone; and
wherein R₃ and R₄ are terminal end groups or structures and wherein R₃ and R₄ do not include ¹⁹F-atoms or ¹⁹F-containing groups.

In yet a fifth instance, the polymer backbone of a ¹⁹F-containing polymer has a polyester structure according to the following formula:

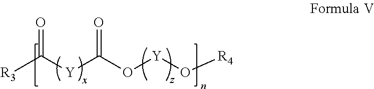

wherein x and z are integer values independently selected from a range from 1 to 10;
wherein n is an integer value in a range from 5 to 2,000;
wherein each Y is independently —CR′R″— and at least one of the R′ or R″ groups is the one or more ¹⁹F-atoms or ¹⁹F-containing groups and the remaining R′ or R″ groups are each independently selected from the group consisting of hydrogen, a halogen, an alkyl group, an aryl group, an alcohol, an amine, an acid (such as a carboxylic acid), a carbonyl group, an ester, and a ketone; and
wherein R₃ and R₄ are terminal groups or structures and wherein the R₃ and R₄ do not include ¹⁹F-atoms or ¹⁹F-containing groups. In some instances of Formula, x and z are preferably 2, such as by condensing ethylene diol and succinic acid either or both including one or more ¹⁹F-atoms or ¹⁹F-containing groups thereon.

In a sixth instance, the polymer backbone of a ¹⁹F-containing polymer has a polyester or polyamide structure according to the following formula:

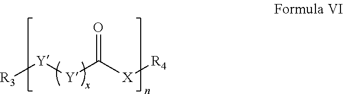

wherein X is —O— or —N(H)—;
wherein z is an integer value in a range from 1 to 10;
wherein n is an integer value in a range from 5 to 2,000;
wherein each Y′ is independently —CR′R″— and at least one of the R′ or R″ groups of either Y′ group is the one or more ¹⁹F-atoms or ¹⁹F-containing groups and the remaining R′ or R″ groups are each independently selected from the group consisting of hydrogen, a halogen, an alkyl group, an aryl group, an alcohol, an amine, an acid (such as a carboxylic acid), a carbonyl group, an ester, and a ketone; and
wherein R₃ and R₄ are terminal groups or structures and wherein the R₃ and R₄ do not include ¹⁹F-atoms or ¹⁹F-containing groups. In some instances of Formula, x and z are preferably 2, such as by condensing ethylene diol and succinic acid either or both including one or more ¹⁹F-atoms or ¹⁹F-containing groups thereon. Such polymers of Formula VI can be formed by ring opening of cyclic lactone or lactam monomers including one or more ¹⁹F-atoms or ¹⁹F-containing groups thereon.

In still a seventh instance, the polymer backbone of a ¹⁹F-containing polymer has a polyamide structure according to the following formula:

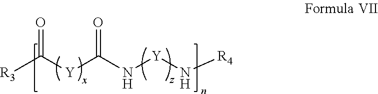

wherein x and z are integer values independently selected from a range from 1 to 10;
wherein n is an integer value in a range from 5 to 2,000;
wherein each Y is independently —CR′R″— and at least one of the R′ or R″ groups is the one or more ¹⁹F-atoms or ¹⁹F-containing groups and the remaining R′ or R″ groups are each independently selected from the group consisting of hydrogen, a halogen, an alkyl group, an aryl group, an alcohol, an amine, an acid (such as a carboxylic acid), a carbonyl group, an ester, and a ketone; and
wherein R₃ and R₄ are terminal groups or structures and wherein the R₃ and R₄ do not include ¹⁹F-atoms or ¹⁹F-containing groups.

For the polymers of the above formulas, n can be in ranges from 5 to 1,750, 5 to 1,500, 5 to 1,250, or 500 to 1,000, as well as individual values or sub-ranges contained within the aforementioned ranges.

In some instances, the ¹⁹F-containing polymer is a homopolymer. In some other instances, the ¹⁹F-containing polymer is a copolymer, such as a random or block/blocky copolymer, and at least one of the R₃ and R₄ terminal groups or structures of the above formulae are polymer segments. The polymer segments may have any suitable structure that does not negatively affect the hydrophilicity and solubility of the ¹⁹F-containing polymer, when attached thereto. In some instances, the polymer segments may be chosen to increase or tune a property, such as hydrophilicity. In some instances, the polymer segments may themselves be formed of more than one type of polymer type. In other words, a polymer segment may be a copolymer.

In some instances, the R₃ and R₄ terminal groups or structures of the above formulae are a targeting and/or active agents (such as a drug or photosensitizer) which are covalently attached to the polymer. Without limitation, the targeting and/or active agent can be selected from a targeting peptide (such as RGD or iRGD), an antibody, a protein, a peptide, an enzyme substrate, a hormone, an antigen, a hapten, an avidin, a streptavidin, biotin, a carbohydrate, an oligosaccharide, a polysaccharide, a nucleic acid, a deoxy nucleic acid, a fragment of DNA, a fragment of RNA, nucleotide triphosphates, acyclo terminator triphosphates, peptide nucleic acid (PNA) biomolecules, a therapeutic agent, a chemotherapeutic agent, a diagnostic agent, a theranostic agent, a photosensitizer (such as IR700 or a phthalocyanine dye), and combinations thereof.

In yet other instances, the R₃ and R₄ terminal groups or structures of the above formulae can be a combination of a targeting and/or active agent on one R group and a polymer segment, such as those described above, on the other R group. In still other instances, the R₃ and R₄ terminal groups or structures of the above formulae are the end groups that are formed upon termination of the growth of the polymer backbone.

Various forms of ¹⁹F-containing polymers are possible, such as where the polymers can have both targeting and active agent(s) covalently bonded thereto, only targeting agent(s) covalently bonded thereto, or only active agent(s) covalently bonded thereto. The targeting and active agents can be those described above. In some instances, one or both of targeting and active agent(s) are not covalently attached to the ¹⁹F-containing polymers and one or both of targeting and active agent(s) are co-administered with the ¹⁹F-containing polymers, as described in the systems below. In still other instances, one or both of targeting and active agent(s) are non-covalently associated to the ¹⁹F-containing polymers and one or both of targeting and active agent(s) are co-administered with the ¹⁹F-containing polymers, as described in the systems below.

In addition to linear polymers, a ¹⁹F-containing polymer may also be branched ¹⁹F-containing polymers, such as multi-armed ¹⁹F-containing polymers. Thus, in some non-limiting instances, a ¹⁹F-containing polymer includes:
a polymer core including a plurality of arms, wherein each arm includes a plurality of polymer repeat units;
wherein at least about 1% and up to about 99% of the polymer repeat units of the plurality include one or more ¹⁹F-atoms or ¹⁹F-containing groups covalently associated thereto; and
wherein the ¹⁹F-containing polymer is hydrophilic and is soluble or substantially soluble in one or more pharmaceutically acceptable carriers. “Substantially soluble,” refers to the ¹⁹F-containing polymer being fully dissolved in the one or more pharmaceutically acceptable carriers at weight percentages in ranges of about 0.1 wt. % to 20 wt. %, 0.1 wt. % to 15 wt. %, 0.1 wt. % to 10 wt. %, or 0.1 wt. % to 5 wt. %, as well as individual values or sub-ranges contained within the aforementioned ranges.

In some instances, in addition to or in place of at least about 1% and up to 99% of the polymer repeat units of the plurality including one or more ¹⁹F-atoms or ¹⁹F-containing groups covalently associated thereto, the polymer repeat units of the plurality include the one or more ¹⁹F-atoms or ¹⁹F-containing groups covalently associated thereto and the molar percentage of fluorine per repeat unit is less than about 75%, 70%, 60%, 50%, 40%, 30%, 20%, 10%, 5%, or 1%; or the molar percentage of fluorine per repeat unit is selected to be in a range from about 1% to 75%, as well as individual values or sub-ranges contained within the aforementioned range. The percentage of fluorine per repeat unit is calculated by the total molar mass of fluorine atom(s) present on each repeat unit divided by the total molar mass of all of the atoms of the repeat unit times 100.

In some instances, the multi-armed ¹⁹F-containing polymer includes a total of 2, 3, 4, 5, 6, 7, 8, or more arms.

In some instances, the polymer core of a multi-armed ¹⁹F-containing polymer is a branched polyethylene imine core. In some instances, the polymer core of a multi-armed ¹⁹F-containing polymer is a mono-, di-, tri- or penta-erythritol, or a glycerol or polyglycerol (up to six repeat units, e.g., hexaglycerol) core.

In some instances, the multi-armed ¹⁹F-containing polymer further includes at least one targeting and/or active agent associated, such as covalently associated, thereto. The at least one targeting and/or active agent can be selected, without limitation, from a targeting peptide (such as RGD or iRGD), an antibody, a protein, a peptide, an enzyme substrate, a hormone, an antigen, a hapten, an avidin, a streptavidin, biotin, a carbohydrate, an oligosaccharide, a polysaccharide, a nucleic acid, a deoxy nucleic acid, a fragment of DNA, a fragment of RNA, nucleotide triphosphates, acyclo terminator triphosphates, peptide nucleic acid (PNA) biomolecules, a therapeutic agent, a chemotherapeutic agent, a diagnostic agent, a theranostic agent, a photosensitizer (such as IR700 or a phthalocyanine dye), and combinations thereof.

In some instances, for the various ¹⁹F-containing polymers described above, whether linear or branched, at least about 1% and up to about 95%, 1% and up to about 90%, 1% and up to about 85%, 1% and up to about 80%, 1% and up to about 75%, 1% and up to about 70%, 1% and up to about 65%, 1% and up to about 60%, 1% and up to about 55%, 1% and up to about 50% of the polymer repeat units of the plurality include one or more ¹⁹F-atoms or ¹⁹F-containing groups covalently associated thereto. In some other instances, at least about 10% and up to about 90%, 10% and up to about 90%, 10% and up to about 85%, 10% and up to about 80%, 10% and up to about 75%, 10% and up to about 70%, 10% and up to about 65%, 10% and up to about 60%, 10% and up to about 55%, 10% and up to about 50% of the polymer repeat units of the plurality include one or more ¹⁹F-atoms or ¹⁹F-containing groups covalently associated thereto. In yet other instances, at least about 10% and up to about 40%, 10% and up to about 35%, 10% and up to about 30%, 10% and up to about 25%, 10% and up to about 20%, 10% and up to about 15% of the polymer repeat units of the plurality include one or more ¹⁹F-atoms or ¹⁹F-containing groups covalently associated thereto. In other instances, at least about 1% and up to about 40%, 10% and up to about 35%, 1% and up to about 30%, 1% and up to about 25%, 1% and up to about 20%, 1% and up to about 15%, or 1% and up to about 10% of the polymer repeat units of the plurality include one or more ¹⁹F-atoms or ¹⁹F-containing groups covalently associated thereto. Individual values or sub-ranges of the aforementioned ranges above are also disclosed.

For the various ¹⁹F-containing polymers described above, whether linear or branched, the one or more ¹⁹F-containing groups can be ¹⁹F-containing alkyl groups, such as —CF₃. In some other instances, the one or more ¹⁹F-containing groups can be selected from trifluoromethyl, pentafluoroethyl, hexafluoropropyl, difluoromethyl, fluoromethyl, 1,1,1-trifluoroethyl, perfluoroisopropyl, tetrafluoroethyl, pentafluoroisopropyl, and combinations thereof. In some instances, the one or more ¹⁹F-containing groups are C₁-C₁₂ alkyl groups wherein at least one and up to all hydrogens thereon are substituted by ¹⁹F.

For the various ¹⁹F-containing polymers described above, whether linear or branched, these polymers can have any suitable molecular weight. In some instances, the ¹⁹F-containing polymer has a weight-average molecular weight in a range from about 500 to 200,000 Daltons, 500 to 175,000 Daltons, 500 to 150,000 Daltons, 500 to 125,000 Daltons, 500 to 100,000 Daltons, 500 to 75,000 Daltons, 500 to 50,000 Daltons, 500 to 25,000 Daltons, 500 to 10,000 Daltons, 500 to 5,000 Daltons, 1,000 to 75,000 Daltons, 1,000 to 50,000 Daltons, 1,000 to 25,000 Daltons, 1,000 to 10,000 Daltons, or 1,000 to 5,000 Daltons, as well as individual values or sub-ranges contained within the aforementioned ranges. In some instances, the ¹⁹F-containing polymer has a hydrodynamic diameter of less than about 50 nm, 40 nm, 30 nm, 20 nm, 10 nm, or 5 nm. In some instances, the ¹⁹F-containing polymer has a hydrodynamic diameter in a range from about 5 nm to 50 nm, as well as individual values or sub-ranges contained within the aforementioned range.

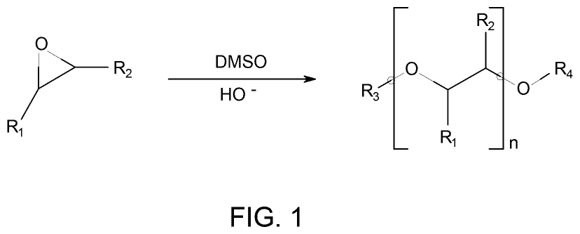

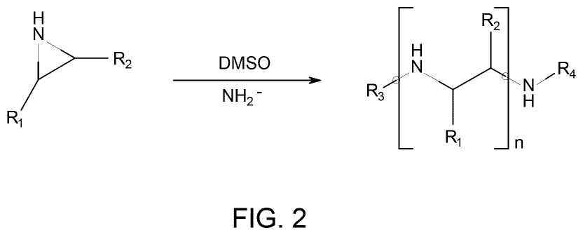

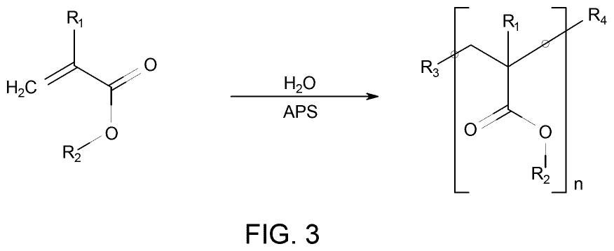

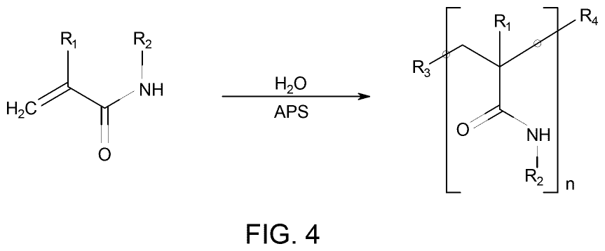

The various ¹⁹F-containing polymers described herein, such as polymers having a structure of any one of the above formulas, can be synthesized using synthetic techniques known in the art of polymer synthesis. Exemplary reaction schemes are shown in FIGS. 1-4. For such synthetic conditions, choice of solvent(s), temperatures (such as to achieve refluxing), atmospheres, work-up conditions, purification conditions, etc.) can be varied or modified from those discussed herein, as appropriate, by the person of ordinary skill in the art of synthetic chemistry, without negatively impacting the polymer synthesis. The methods may include additional steps, which are not otherwise specified, that may be involved in the syntheses which include, but are not limited to, precipitation of a product, washing the reaction mixture, extracting the reaction mixture, separation of the product from a mixture (e.g. by filtration), drying the product, and combinations thereof. Further processing steps that may be involved include stirring, heating and/or cooling the reaction mixture. Intermediate, crude, and purified products can be characterized using any suitable characterization method known to the skilled person. In some instances, such methods include, but are not limited to, NMR, mass spectrometry, chromatography, elemental analysis, and/or other techniques.

### A. Pharmaceutical Compositions

Also disclosed herein are pharmaceutical compositions which include the ¹⁹F-containing polymers described herein. The pharmaceutical composition includes an effective amount of the ¹⁹F-containing polymers for purposes of ¹⁹F magnetic resonance imaging.

In one non-limiting instance, a pharmaceutical composition includes:
a plurality of the ¹⁹F-containing polymers, as described herein; and
one or more pharmaceutically acceptable carriers;
wherein the plurality of the ¹⁹F-containing polymers are solubilized or substantially solubilized in the one or more pharmaceutically acceptable carriers. “Substantially soluble,” refers to the ¹⁹F-containing polymer being fully dissolved in one or more pharmaceutically acceptable carriers at standard temperature and pressure and at weight percentages in ranges of about 0.1 wt. % to 20 wt. %, 0.1 wt. % to 15 wt. %, 0.1 wt. % to 10 wt. %, or 0.1 wt. % to 5 wt. %, as well as individual values or sub-ranges contained within the aforementioned ranges.

Reference to a plurality of the ¹⁹F-containing polymers is understood to encompass instances where the plurality includes only one type of ¹⁹F-containing polymer, as well as instances where combinations of two or more types of different ¹⁹F-containing polymers are present in the pharmaceutical composition.

In some instances, the one or more pharmaceutically acceptable carriers are selected from water, ethanol, polyethylene glycol, propylene glycol, and combinations thereof. Other suitable carriers, such as aqueous carriers, are known. In some instances, the pharmaceutical composition is an aqueous pharmaceutical composition. In some instances, the composition further includes one or more excipients that can provide a desired tonicity or osmolarity to the composition. Excipients suitable for providing osmolarity or tonicity include, without limitation, sugars including but not limited to mannitol, dextrose or glucose and salts including but not limited to sodium chloride or sodium phosphate.

In some instances, the ¹⁹F-containing polymer(s) are present in a concentration ranging from about 0.1 wt. % to 15 wt. %, 0.1 wt. % to 10 wt. %, or 0.1 wt. % to 5 wt. % of the total weight of the pharmaceutical composition, as well as individual values and sub-ranges contained within the aforementioned ranges.

In some cases, pharmaceutical compositions containing an effective amount of the ¹⁹F-containing polymer(s), can optionally include one or more additional therapeutic, prophylactic, and/or diagnostic agents therein. Pharmaceutical compositions can be for administration by parenteral (intramuscular, intraperitoneal, intradermal, intravenous (IV), or subcutaneous injection) or enteral routes of administration and can be formulated in dosage forms appropriate for each route of administration.

i. Parenteral Compositions

Pharmaceutical compositions containing the ¹⁹F-containing polymer(s) can be administered in an aqueous solution, by parenteral injection. Such compositions may also be in the form of a suspension or emulsion. In general, pharmaceutical compositions are provided including effective amounts of the ¹⁹F-containing polymer(s) and optionally include other pharmaceutically acceptable diluents, preservatives, solubilizers, emulsifiers, adjuvants and/or carriers. Such compositions can include diluents sterile water, buffered saline of various buffer content (e.g., Tris-HCl, acetate, phosphate), pH and ionic strength; and optionally, one or more additives such as detergents or solubilizing agents (e.g., TWEEN® 20, TWEEN® 80 also referred to as polysorbate 20 or 80), antioxidants (e.g., ascorbic acid, sodium metabisulfite), preservatives (e.g., Thimersol, benzyl alcohol), metal scavenging agents (e.g., ethylenediaminetetraacetic acid (EDTA) and/or bulking substances (e.g., lactose, mannitol).

ii. Dosage Units

In some instances, the pharmaceutical compositions are in the form of dosage units which include a powder of the ¹⁹F-containing polymer(s) that can be reconstituted with one or more pharmaceutically acceptable carriers. Either one or both of the powder and the carriers may optionally include one or more additional active agents, such as those described therein. Upon reconstitution, the composition can be used as an injectable ¹⁹F MR imaging composition. In some instances, following reconstitution, the mass concentration of the ¹⁹F-containing polymer(s) in the composition may be in a range from about 0.1 to 150 mg/mL, 0.1 to 125 mg/mL, 0.1 to 100 mg/mL, 0.1 to 50 mg/mL, 0.1 to 40 mg/mL, 0.1 to 30 mg/mL, 0.1 to 20 mg/mL, 0.1 to 10 mg/mL, 0.1 to 5 mg/mL, 0.5 to 50 mg/mL, 0.5 to 40 mg/mL, 0.5 to 30 mg/mL, 0.5 to 20 mg/mL, 0.5 to 10 mg/mL, 0.5 to 5 mg/mL, 1 to 50 mg/mL, 1 to 40 mg/mL, 1 to 30 mg/mL, 1 to 20 mg/mL, 1 to 10 mg/mL, or 1 to 5 mg/mL, as well as individual values or sub-ranges contained within the aforementioned ranges. In such instances, the ¹⁹F-containing polymer(s) may be provided in containers or vials containing a powder of the polymer(s) in amounts needed to provide or prepare a desired concentration when reconstituted with the one or more pharmaceutically acceptable carriers. In some instances, the ¹⁹F-containing polymer(s) may undergo further processing with excipients to create a dry powder, where such excipients can provide a desired tonicity or osmolarity after reconstitution with a pharmaceutically acceptable carrier(s) prior to administration to a subject. Excipients suitable for providing osmolarity or tonicity include, without limitation, sugars including but not limited to mannitol, dextrose or glucose and salts including but not limited to sodium chloride or sodium phosphate.

iv. Optional Therapeutic, Prophylactic or Diagnostic Agents

In some cases, the pharmaceutical compositions can optionally include one or more additional active agents. Therefore, in some forms, the pharmaceutical composition includes the ¹⁹F-containing polymer(s), in addition to at least one of a therapeutic, prophylactic, and/or diagnostic agents. The additional active agents can be covalently associated to the ¹⁹F-containing polymer(s) or the active agents may be provided as a separate composition or combination composition (i.e., in a single formulation wherein the active agents are not covalently associated to the polymer(s)) for co-administration with the polymer(s).

Non-limiting examples of therapeutic, prophylactic, or diagnostic agents can include corticosteroids, anti-angiogenesis agents, antibiotics, antioxidants, anti-viral agents, anti-fungal agents, anti-inflammatory agents, immunosuppressant agents, anti-allergic agents, and combinations thereof. In some instances, such agents may also include targeting agents, such as a protein, a peptide, an enzyme substrate, a hormone, an antibody, an antigen, a hapten, an avidin, a streptavidin, biotin, a carbohydrate, an oligosaccharide, a polysaccharide, a nucleic acid, a deoxy nucleic acid, a fragment of DNA, a fragment of RNA, nucleotide triphosphates, acyclo terminator triphosphates, peptide nucleic acid (PNA) biomolecules, and combinations thereof. The amount of any additional therapeutic, prophylactic, or diagnostic agents generally depends on the severity of the diseases and/or disorders to be treated. Specific dosages can be readily determined by those of skill in the art. See Ansel, Howard C. et al., Pharmaceutical Dosage Forms and Drug Delivery Systems (6th ed.) Williams and Wilkins, Malvern, PA (1995).

### B. Systems Including ¹⁹F-Containing Polymers

In some instances, a pharmaceutical composition described herein can be combined with one or more additional compositions to form a system. For example, in one non-limiting instance, such a system can include:
a pharmaceutical composition including:
   a plurality of the ¹⁹F-containing polymers as described herein; and
   one or more pharmaceutically acceptable carriers;
   wherein the plurality of the ¹⁹F-containing polymers are solubilized or substantially solubilized in the one or more pharmaceutically acceptable carriers;
and
   at least one targeting composition including:
   a plurality of agents for targeted delivery of the plurality of the ¹⁹F-containing polymers of the pharmaceutical composition to a target tissue in a subject.

The pharmaceutical composition is described in detail in Section IIA above.

In some instances, the agents for targeted delivery present in the targeting composition are selected from a protein, a peptide, an enzyme substrate, a hormone, an antibody, an antigen, a hapten, an avidin, a streptavidin, biotin, a carbohydrate, an oligosaccharide, a polysaccharide, a nucleic acid, a deoxy nucleic acid, a fragment of DNA, a fragment of RNA, nucleotide triphosphates, acyclo terminator triphosphates, peptide nucleic acid (PNA) biomolecules, and combinations thereof. In some instances, agents for targeted delivery target a tissue selected from a tumor, an organ or soft mass tissue.

The pharmaceutical composition and the targeting composition may be administered in any suitable order such that the agents for targeted delivery cause and result in the plurality of the ¹⁹F-containing polymers of the pharmaceutical composition targeting and being delivered to a target tissue in a subject once administered. In some instances, the targeting composition is administered first followed by the pharmaceutical composition. In other instances, the two compositions are co-administered simultaneously.

In some instances, the targeting composition optionally includes one or more additional active agents, such as therapeutic, prophylactic, and/or diagnostic agents. Non-limiting examples of therapeutic, prophylactic, or diagnostic agents can include corticosteroids, anti-angiogenesis agents, antibiotics, antioxidants, anti-viral agents, anti-fungal agents, anti-inflammatory agents, immunosuppressant agents, anti-allergic agents, and combinations thereof. The amount of any additional therapeutic, prophylactic, or diagnostic agents generally depends on the severity of the diseases and/or disorders to be treated. Specific dosages can be readily determined by those of skill in the art. See Ansel, Howard C. et al., Pharmaceutical Dosage Forms and Drug Delivery Systems (6th ed.) Williams and Wilkins, Malvern, PA (1995).

The targeting composition includes a plurality of agents in a suitable concentration effective for targeted delivery of the plurality of the ¹⁹F-containing polymers wherein the targeting agents are present in one or more pharmaceutically acceptable carriers and optionally one or more pharmaceutically acceptable excipients. In some instances, the concentration of agents for targeted delivery in the targeting composition is based on the concentration/amount of the ¹⁹F-containing polymers in the pharmaceutical composition. In some instances, for example, the concentration of agents for targeted delivery in the targeting composition is about 0.1 wt. % to 75 wt. %, 0.1 wt. % to 70 wt. %, 0.1 wt. % to 65 wt. %, 0.1 wt. % to 60 wt. %, 0.1 wt. % to 55 wt. %, 0.1 wt. % to 50 wt. %, 0.1 wt. % to 45 wt. %, 0.1 wt. % to 40 wt. %, 0.1 wt. % to 35 wt. %, 0.1 wt. % to 30 wt. %, 0.1 wt. % to 25 wt. %, 0.1 wt. % to 20 wt. %, 0.1 wt. % to 15 wt. %, 0.1 wt. % to 10 wt. %, 0.1 wt. % to 5 wt. %, or 0.1 wt. % to 1 wt. % of the amount of the ¹⁹F-containing polymers in the pharmaceutical composition, as well as individual values and sub-ranges contained within the aforementioned ranges. In some instances, the one or more pharmaceutically acceptable carriers are selected from water, ethanol, polyethylene glycol, propylene glycol, and combinations thereof. Other suitable carriers, such as aqueous carriers, are known. In some instances, the pharmaceutical composition is an aqueous pharmaceutical composition.

Thus, for such systems it is possible for the plurality of the ¹⁹F-containing polymers to include or exclude targeting and/or active agent(s) covalently associated thereto, as previously described, and the targeting composition includes the targeting agent(s), and optionally active agent(s), that are co-administered with the pharmaceutical composition.

### III. Methods of Using ¹⁹F-Containing Polymers as Magnetic Resonance Imaging Agents

The ¹⁹F-containing polymers described herein can be used as a diagnostic and imaging tool for various biological applications. Of particular interest is their use in imaging in the area of oncology, such as to treat or ameliorate a cancer in a subject in need thereof. In some instances, the cancer is selected from, but not limited to, solid cancers such as breast, lung, colorectal, pancreatic, liver, kidney, prostate, brain, head, and neck cancers. In addition to cancers, the ¹⁹F-containing polymer imaging agents can be used to image other conditions or diseases that have a specific marker that can be targeted, such as, but not limited to inflammation, a tissue injury, and/or a specific organ defect/abnormality.

For the various ¹⁹F-containing polymers and compositions thereof, it is understood that in some instances the ¹⁹F-containing polymers are conjugated to one or more active agents (such as a targeting agent and/or a drug) and can be administered as part of a pharmaceutical composition alone and without co-administration with any other compositions. In other instances, the ¹⁹F-containing polymers are conjugated to one or more active agents (such as a targeting agent and/or a drug) and are co-administered with another composition that also active agents (such as a targeting agent and/or a drug). In still other instances, the ¹⁹F-containing polymers do not have any active agents bonded thereto and are co-administered with another composition including one or more active agents (such as a targeting agent and/or a drug). In other words, a targeting agent and/or drug may be covalently attached to a ¹⁹F-containing polymer or these agents may be separate from the polymer and co-administered therewith.

In one non-limiting instance, a method of imaging a target tissue in a subject includes the steps of:
(i) administering a pharmaceutical composition described herein to a subject;
(ii) allowing an effective period of time for the plurality of the ¹⁹F-containing polymers of the pharmaceutical composition to accumulate in the target tissue of the subject; and
(iii) imaging by ¹⁹F magnetic resonance imaging the plurality of the ¹⁹F-containing polymers following step (ii);
   wherein the imaging permits visualizing the target tissue and optionally may be used to analyze size, shape, position, and composition of the target tissue.

For the above method, the pharmaceutical composition includes a plurality of the ¹⁹F-containing polymers as described above and, in at least some instances, at least some of the ¹⁹F-containing polymers include a targeting and/or active agent covalently associated thereto. The presence of a targeting agent can be used to target the ¹⁹F-containing polymers to the target tissue.

In another non-limiting instance, a method of imaging a target tissue in a subject includes the steps of:
(i′) administering a system described herein (i.e., including a pharmaceutical compositions and a targeting composition) to a subject;
(ii′) allowing an effective period of time for the plurality of the ¹⁹F-containing polymers of the pharmaceutical composition to accumulate in a target tissue of the subject; and
(iii′) imaging by ¹⁹F magnetic resonance imaging the plurality of the ¹⁹F-containing polymers following step (ii′);
   wherein the imaging permits visualizing the target tissue and optionally may be used to analyze size, shape, position, and composition of the target tissue.

For the above system method, the pharmaceutical composition and the targeting composition may be administered in any suitable order such that the agents for targeted delivery in the targeting composition cause and result in the plurality of the ¹⁹F-containing polymers of the pharmaceutical composition targeting and being delivered to a target tissue in a subject once administered. In some instances, the targeting composition is administered first followed by the pharmaceutical composition. In other instances, the two compositions are co-administered simultaneously, either by injecting the compositions at the same time or mixing the compositions and injecting the mixture thereof. As previously noted, for methods involving administration of the systems described it is possible for the plurality of the ¹⁹F-containing polymers to include or exclude targeting and/or active agent(s) covalently associated thereto, such that the targeting composition includes the targeting agent(s), and optionally active agent(s), which are co-administered with the pharmaceutical composition.

In still another non-limiting instance, a method of treating or ameliorating a disease or condition in a subject in need thereof includes the steps of:
(a) administering a pharmaceutical composition or a system, such as those described herein, to a subject;
(b) allowing an effective period of time for the plurality of the ¹⁹F-containing polymers of the pharmaceutical composition to accumulate in a target tissue of the subject; and
(c) imaging by ¹⁹F magnetic resonance imaging the plurality of the ¹⁹F-containing polymers following step (b);
   wherein the imaging permits visualizing the target tissue and optionally may be used to analyze size, shape, position, and composition of the target tissue;
wherein the ¹⁹F-containing polymers include at least one active agent associated thereto selected from the group consisting of a therapeutic agent, a chemotherapeutic agent, a photosensitizer (such as IR700 or a phthalocyanine dye), and combinations thereof.

In some instances of the various methods described herein, the ¹⁹F-containing polymers of the pharmaceutical composition also include at least one targeting and/or active agent associated, such as covalently associated, thereto. Such agents are as previously described above.

For the various methods described, the target tissue can be selected from a tumor, an organ, and/or soft tissues of the subject.

For the above method where the ¹⁹F-containing polymers include at least one active agent associated thereto, when the at least one active agent is a photosensitizer (such as IR700 or a phthalocyanine dye), the method further includes the step of:
(d) administering a light dosage to activate the photosensitizer.

In some instances, the light dosage kills the target tissue. In some instances, the light dosage is less than 1 KJ/cm², and wherein the light dosage is applied from a light source selected from an LED, a continuous laser, a pulsed laser, or combinations thereof. In some instances, the light dosage is between 100 and 400 J/cm².

For the various methods described, the administering step is typically via intravenous injection.

For the methods described herein, ¹⁹F magnetic resonance imaging can be achieved using known MRI systems, an appropriate ¹⁹F probe, and skills known in the relevant art of magnetic resonance imaging. In some instances, high-field Systems (≥˜3 Tesla) are preferred as higher magnetic field strengths can improve signal-to-noise ratio (SNR), as ¹⁹F imaging may have lower sensitivity compared to 1H imaging. High fields can also allow better spectral resolution for distinguishing multiple ¹⁹F environments in the imaging study. Various pulse sequences may be used for ¹⁹F magnetic resonance imaging, such as: Gradient-Echo (GRE); Spin-Echo (SE); Fast Imaging techniques (such as Echo-planar imaging (EPI) or balanced steady-state free precession (bSSFP)); and Multi-Echo Sequences (which enable simultaneous acquisition of multiple ¹⁹F species with different relaxation properties). Regarding relaxation properties, such as T₁ and T₂, adjustments to pulse repetition time (TR) and echo time (TE) may be made as needed, to maximize SNR by enhancing signal recovery and/or TE can be used to minimize T₂ decay. In some instances, imaging systems may employ the use of signal averaging for improved SNR given the lower sensitivity of ¹⁹F-based imaging agents.

### IV. Kits

Kits useful for practicing the methods described above, and the various pharmaceutical and other compositions used therein, are also disclosed herein. For example, kits for ¹⁹F-based imaging applications can include at least one or more of the pharmaceutical compositions or systems including the ¹⁹F-containing polymer(s) and optionally a targeting composition, each as described above.

In one non-limiting instance, a kit includes a vial of a composition including the ¹⁹F-containing polymer(s) in either liquid form (i.e., as a pharmaceutical composition) or in solid form (i.e., as a dosage unit that is reconstituted before use with a pharmaceutically acceptable carrier to form a pharmaceutical composition). Examples of such compositions are described in detail above.

In another non-limiting instance, a kit includes two separate vials where one vial includes the ¹⁹F-containing polymer(s) in either liquid form (i.e., as a pharmaceutical composition) or in solid form (i.e., as a dosage unit that is reconstituted before use with a pharmaceutically acceptable carrier to form a pharmaceutical composition) and the other vial includes a second composition, such as a targeting composition. Examples of such compositions are described in detail above. The compositions can be mixed and administered as single mixture or can sequentially administered.

### EXAMPLES

### Example 1: ¹⁹F NMR Data of 3,3,3-trifluoropropane-1,2-diol

### Materials and Methods:

3,3,3-trifluoropropane-1,2-diol (CAS. No. 431-39-0) was obtained from Chem Scene and subjected to ¹⁹F NMR analysis.

More specifically, the T₂ of the fluorinated compound was obtained by dissolving the compound in D₂O at 25° C. and 11.7 T using the following general model:

### General Model:

$$T 2\_ 19 F ( x ) = a * exp ( - x / T 2 )$$

Coefficients (with 95% Confidence Bounds):

$$T 2 = 0.1629 ( 0.1571 , 0.1686 )$$

$$a = 3.414 ( 3.25 , 3.579 )$$

### DISCUSSION

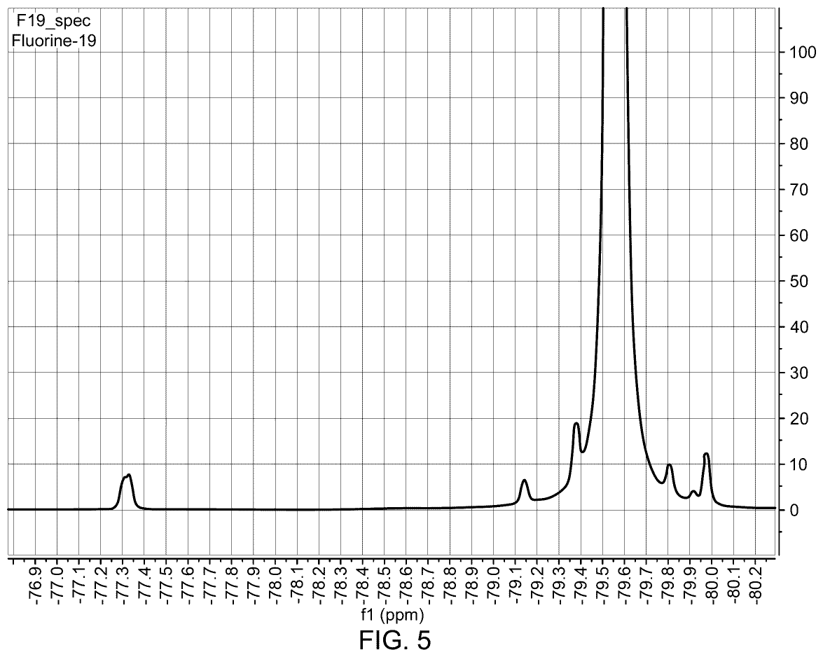

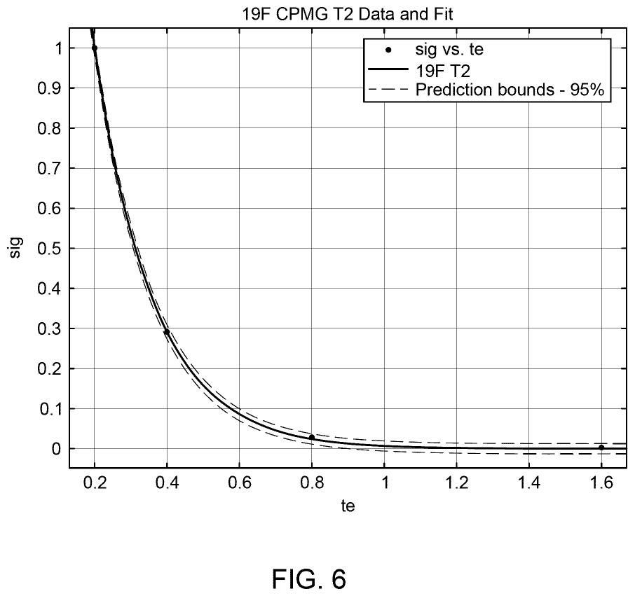

¹⁹F NMR of 3,3,3-trifluoropropane-1,2-diol was taken as a model monomer unit which may form part of a ¹⁹F-containing polymer. The ¹⁹F NMR spectrum is shown in FIG. 5. FIG. 6 shows the fitted T₂ based on the NMR data. There are two main resonances that overlapped.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific instances of the invention described herein. Such equivalents are intended to be encompassed by the following claims.

1. A ¹⁹F-containing polymer comprising:
a polymer backbone formed of a plurality of polymer repeat units;
wherein at least about 1% and up to 99% of the polymer repeat units of the plurality comprise one or more ¹⁹F-atoms or ¹⁹F-containing groups covalently associated thereto; and
wherein the ¹⁹F-containing polymer is hydrophilic and is soluble or substantially soluble in one or more pharmaceutically acceptable carriers.

2. The ¹⁹F-containing polymer of claim 1, wherein the polymer backbone has a structure according to the following formula: 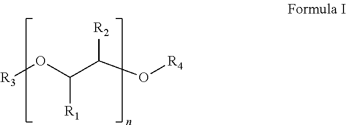
wherein n is an integer value in a range from 5 to 2,000;
wherein at least some, but not all, of the R₁ and R₂ groups are the one or more ¹⁹F-atoms or ¹⁹F-containing groups and the remaining R₁ and R₂ groups are each independently selected from the group consisting of hydrogen, a halogen, an alkyl group, an aryl group, an alcohol, an amine, an acid (such as a carboxylic acid), a carbonyl group, an ester, and a ketone; and
wherein R₃ and R₄ are terminal end groups or structures and wherein the R₃ and R₄ do not comprise ¹⁹F-atoms or ¹⁹F-containing groups.

3. The ¹⁹F-containing polymer of claim 1, wherein the polymer backbone has a structure according to the following formula: 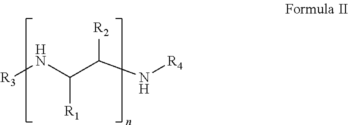
wherein n is an integer value in a range from 5 to 2,000;
wherein at least some, but not all, of the R₁ and R₂ groups are the one or more ¹⁹F-atoms or ¹⁹F-containing groups and the remaining R₁ and R₂ groups are each independently selected from the group consisting of hydrogen, a halogen, an alkyl group, an aryl group, an alcohol, an amine, an acid (such as a carboxylic acid), a carbonyl group, an ester, and a ketone; and
wherein R₃ and R₄ are terminal end groups or structures and wherein the R₃ and R₄ do not comprise ¹⁹F-atoms or ¹⁹F-containing groups.

4. The ¹⁹F-containing polymer of claim 1, wherein the polymer backbone has a structure according to the following formula: 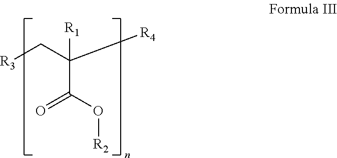
wherein n is an integer value in a range from 5 to 2,000;
wherein at least some, but not all, of the R₁ and R₂ groups are the one or more ¹⁹F-atoms or ¹⁹F-containing groups and the remaining R₁ and R₂ groups are each independently selected from the group consisting of hydrogen, a halogen, an alkyl group, an aryl group, an alcohol, an amine, an acid (such as a carboxylic acid), a carbonyl group, an ester, and a ketone; and
wherein R₃ and R₄ are terminal groups or structures and wherein the R₃ and R₄ do not comprise ¹⁹F-atoms or ¹⁹F-containing groups.

5. The ¹⁹F-containing polymer of claim 1, wherein the polymer backbone has a structure according to the following formula: 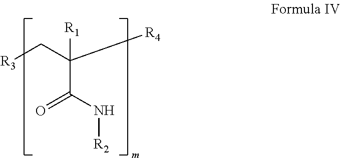
wherein n is an integer value in a range from 5 to 2,000;
wherein at least some, but not all, of the R₁ and R₂ groups are the one or more ¹⁹F-atoms or ¹⁹F-containing groups and the remaining R₁ and R₂ groups are each independently selected from the group consisting of hydrogen, a halogen, an alkyl group, an aryl group, an alcohol, an amine, an acid (such as a carboxylic acid), a carbonyl group, an ester, and a ketone; and
wherein R₃ and R₄ are terminal groups or structures and wherein the R₃ and R₄ do not comprise ¹⁹F-atoms or ¹⁹F-containing groups.

6. The ¹⁹F-containing polymer of claim 1, wherein the polymer backbone has a structure according to the following formula: 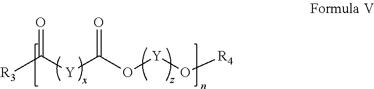
wherein x and z are integer values independently selected from a range from 1 to 10;
wherein n is an integer value in a range from 5 to 2,000;
wherein each Y is independently —CR′R″— and at least one of the R′ or R″ groups is the one or more ¹⁹F-atoms or ¹⁹F-containing groups and the remaining R′ or R″ groups are each independently selected from the group consisting of hydrogen, a halogen, an alkyl group, an aryl group, an alcohol, an amine, an acid (such as a carboxylic acid), a carbonyl group, an ester, and a ketone; and
wherein R₃ and R₄ are terminal groups or structures and wherein the R₃ and R₄ do not include ¹⁹F-atoms or ¹⁹F-containing groups.

7. The ¹⁹F-containing polymer of claim 1, wherein the polymer backbone has a structure according to the following formula: 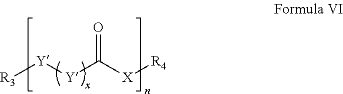
wherein X is —O— or —N(H)—;
wherein z is an integer value in a range from 1 to 10;
wherein n is an integer value in a range from 5 to 2,000;
wherein each Y′ is independently —CR′R″— and at least one of the R′ or R″ groups of either Y′ group is the one or more ¹⁹F-atoms or ¹⁹F-containing groups and the remaining R′ or R″ groups are each independently selected from the group consisting of hydrogen, a halogen, an alkyl group, an aryl group, an alcohol, an amine, an acid (such as a carboxylic acid), a carbonyl group, an ester, and a ketone; and
wherein R₃ and R₄ are terminal groups or structures and wherein the R₃ and R₄ do not include ¹⁹F-atoms or ¹⁹F-containing groups.

8. The ¹⁹F-containing polymer of claim 1, wherein the polymer backbone has a structure according to the following formula: 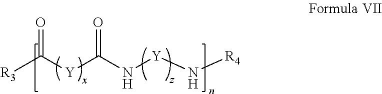
wherein x and z are integer values independently selected from a range from 1 to 10;
wherein n is an integer value in a range from 5 to 2,000;
wherein each Y is independently —CR′R″— and at least one of the R′ or R″ groups is the one or more ¹⁹F-atoms or ¹⁹F-containing groups and the remaining R′ or R″ groups are each independently selected from the group consisting of hydrogen, a halogen, an alkyl group, an aryl group, an alcohol, an amine, an acid (such as a carboxylic acid), a carbonyl group, an ester, and a ketone; and
wherein R₃ and R₄ are terminal groups or structures and wherein the R₃ and R₄ do not include ¹⁹F-atoms or ¹⁹F-containing groups.

9. The ¹⁹F-containing polymer of claim 1, wherein the polymer backbone has a structure according to any one of Formulas I′, II′, III′, or IV′ as described in the specification.

10. The ¹⁹F-containing polymer of claim 1, wherein the one or more ¹⁹F-containing groups are ¹⁹F-containing alkyl groups, such as —CF₃.

11. The ¹⁹F-containing polymer of claim 1, wherein the ¹⁹F-containing polymer is a homopolymer.

12. The ¹⁹F-containing polymer of claim 2, wherein the ¹⁹F-containing polymer is a copolymer, such as a random or block copolymer, and at least one of the R₃ and R₄ terminal groups or structures are polymers.

13. The ¹⁹F-containing polymer of claim 2, wherein at least one of the R₃ and R₄ terminal groups or structures is a targeting and/or active agent.

14. The ¹⁹F-containing polymer of claim 13, wherein the targeting and/or active agent is selected from a targeting peptide (such as RGD or iRGD), an antibody, a protein, a peptide, an enzyme substrate, a hormone, an antigen, a hapten, an avidin, a streptavidin, biotin, a carbohydrate, an oligosaccharide, a polysaccharide, a nucleic acid, a deoxy nucleic acid, a fragment of DNA, a fragment of RNA, nucleotide triphosphates, acyclo terminator triphosphates, peptide nucleic acid (PNA) biomolecules, a therapeutic agent, a chemotherapeutic agent, a diagnostic agent, a theranostic agent, a photosensitizer (such as IR700 or a phthalocyanine dye), and combinations thereof.

15. A ¹⁹F-containing polymer comprising:
a polymer core comprising a plurality of arms, wherein each arm comprises a plurality of polymer repeat units;
wherein at least about 1% and up to 99% of the polymer repeat units of the plurality comprise one or more ¹⁹F-atoms or ¹⁹F-containing groups covalently associated thereto; and
wherein the ¹⁹F-containing polymer is hydrophilic and is soluble or substantially soluble in one or more pharmaceutically acceptable carriers.

16. The ¹⁹F-containing polymer of claim 15, comprising a total of 2, 3, 4, 5, 6, 7, 8, or more arms.

17. (canceled)

18. The ¹⁹F-containing polymer of claim 15, wherein the polymer core is a branched polyethylene imine core.

19-20. (canceled)

21. The ¹⁹F-containing polymer of claim 1, wherein the ¹⁹F-containing polymer has a weight-average molecular weight in a range from about 500 to 200,000 Daltons; and optionally wherein the ¹⁹F-containing polymer has a hydrodynamic diameter of less than about 50 nm, 40 nm, 30 nm, 20 nm, 10 nm, or 5 nm.

22. A pharmaceutical composition comprising:
a plurality of the ¹⁹F-containing polymers, wherein each polymer of the plurality comprises a polymer backbone formed of a plurality of polymer repeat units, wherein at least about 1% and up to 99% of the polymer repeat units of the plurality of polymer repeat units comprise one or more ¹⁹F-atoms or ¹⁹F-containing groups covalently associated thereto, and wherein the polymer is hydrophilic; and
one or more pharmaceutically acceptable carriers;
wherein the plurality of the ¹⁹F-containing polymers are solubilized or substantially solubilized in the one or more pharmaceutically acceptable carriers.

23. (canceled)

24. A method of imaging a target tissue in a subject, the method comprising the steps of:
(i) administering a pharmaceutical composition to the subject, the pharmaceutical composition comprising:
a plurality of the ¹⁹F-containing polymers, wherein each polymer of the plurality comprises a polymer backbone formed of a plurality of polymer repeat units, wherein at least about 1% and up to 99% of the polymer repeat units of the plurality of polymer repeat units comprise one or more ¹⁹F-atoms or ¹⁹F-containing groups covalently associated thereto, and wherein the polymer is hydrophilic; and
one or more pharmaceutically acceptable carriers;
wherein the plurality of the ¹⁹F-containing polymers are solubilized or substantially solubilized in the one or more pharmaceutically acceptable carriers;
(ii) allowing an effective period of time for the plurality of the ¹⁹F-containing polymers of the pharmaceutical composition to accumulate in the target tissue of the subject; and
(iii) imaging by ¹⁹F magnetic resonance imaging the plurality of the ¹⁹F-containing polymers following step (ii);
wherein the imaging permits visualizing the target tissue and optionally may be used to analyze size, shape, position, and composition of the target tissue.

25. A system comprising:
a pharmaceutical composition comprising:
a plurality of the ¹⁹F-containing polymers, wherein each polymer of the plurality comprises a polymer backbone formed of a plurality of polymer repeat units, wherein at least about 1% and up to 99% of the polymer repeat units of the plurality of polymer repeat units comprise one or more ¹⁹F-atoms or ¹⁹F-containing groups covalently associated thereto, and wherein the polymer is hydrophilic; and
one or more pharmaceutically acceptable carriers;
wherein the plurality of the ¹⁹F-containing polymers are solubilized or substantially solubilized in the one or more pharmaceutically acceptable carriers; and
a targeting composition comprising:
a plurality of agents for targeted delivery of the plurality of the ¹⁹F-containing polymers of the pharmaceutical composition to a target tissue in a subject.

26. (canceled)

27. A method of imaging a target tissue in a subject, the method comprising the steps of:
(i′) administering a system to the subject, wherein the system comprises:
a pharmaceutical composition comprising:
a plurality of the ¹⁹F-containing polymers, wherein each polymer of the plurality comprises a polymer backbone formed of a plurality of polymer repeat units, wherein at least about 1% and up to 99% of the polymer repeat units of the plurality of polymer repeat units comprise one or more ¹⁹F-atoms or ¹⁹F-containing groups covalently associated thereto, and wherein the polymer is hydrophilic; and
one or more pharmaceutically acceptable carriers;
wherein the plurality of the ¹⁹F-containing polymers are solubilized or substantially solubilized in the one or more pharmaceutically acceptable carriers; and
a targeting composition comprising:
a plurality of agents for targeted delivery of the plurality of the ¹⁹F-containing polymers of the pharmaceutical composition to a target tissue in a subject;
(ii′) allowing an effective period of time for the plurality of the ¹⁹F-containing polymers of the pharmaceutical composition to accumulate in a target tissue of the subject; and
(iii′) imaging by ¹⁹F magnetic resonance imaging the plurality of the ¹⁹F-containing polymers following step (ii′);
wherein the imaging permits visualizing the target tissue and optionally may be used to analyze size, shape, position, and composition of the target tissue.

28. (canceled)

29. A method of treating or ameliorating a disease in a subject in need thereof, the method comprising the steps of:
(a) administering a pharmaceutical composition, wherein the pharmaceutical composition comprises:
a pharmaceutical composition comprising:
a plurality of the ¹⁹F-containing polymers, wherein each polymer of the plurality comprises a polymer backbone formed of a plurality of polymer repeat units, wherein at least about 1% and up to 99% of the polymer repeat units of the plurality of polymer repeat units comprise one or more ¹⁹F-atoms or ¹⁹F-containing groups covalently associated thereto, and wherein the polymer is hydrophilic; and
one or more pharmaceutically acceptable carriers;
wherein the plurality of the ¹⁹F-containing polymers are solubilized or substantially solubilized in the one or more pharmaceutically acceptable carriers; and
a targeting composition comprising:
a plurality of agents for targeted delivery of the plurality of the ¹⁹F-containing polymers of the pharmaceutical composition to a target tissue in a subject;
(b) allowing an effective period of time for the plurality of the ¹⁹F-containing polymers of the pharmaceutical composition to accumulate in a target tissue of the subject; and
(c) imaging by ¹⁹F magnetic resonance imaging the plurality of the ¹⁹F-containing polymers following step (b);
wherein the imaging permits visualizing the target tissue and optionally may be used to analyze size, shape, position, and composition of the target tissue;
wherein the ¹⁹F-containing polymers comprise at least one active agent associated thereto selected from the group consisting of a therapeutic agent, a chemotherapeutic agent, a photosensitizer (such as IR700 or a phthalocyanine dye), and combinations thereof.

30-35. (canceled)
